(19) United States
(12) Patent Application Publication
(10) Pub. No.: US 2012/0266905 A1
(43) Pub. Date: Oct. 25, 2012

(54) ADHERING ORNAMENTAL PARTICLES TO A NAIL

(76) Inventor: Amanda Best, Cedar Hills, UT (US)

(21) Appl. No.: 13/455,889

(22) Filed: Apr. 25, 2012

Related U.S. Application Data
(60) Provisional application No. 61/478,782, filed on Apr. 25, 2011.

(51) Int. Cl. A45D 29/11; A61Q 3/02; A61K 8/18

(52) U.S. Cl. 132/200; 132/73.5

(57) ABSTRACT

Adhering ornamental particles to a nail comprises: applying a number of base layers of an adhesive lacquer to the nail; disposing a layer of the ornamental particles over the a number of base layers of the adhesive lacquer; covering the layer of ornamental particles with a number of upper layers of the adhesive lacquer; and covering the upper layer of the adhesive lacquer with a number of layers of a sealant material.

### RELATED DOCUMENTS

The present application claims benefit under 35 U.S.C. §119(e) to provisional patent application No. 61/478,782 filed Apr. 25, 2011. This application is herein incorporated by reference in its entirety.

### BACKGROUND

The practice of decorating fingernails and toenails is extremely popular in modern culture. The application of nail polish or artificial nails to the surface of a natural fingernail or toenail provides the advantage of enhancing the aesthetic appearance of the nail while protecting the nail from damage. While many aesthetic nail treatments can be done at home, other nail treatments have traditionally only been available at salons.

One particular nail treatment trend that has gained substantial momentum in recent years is that of “glitter nails,” in which a layer of reflective particles is affixed to one's toenails, thereby causing the toenails to sparkle in an aesthetically pleasing manner. Some people attempt to achieve this effect using a dispersion of glitter within ordinary nail polish. Unfortunately, ordinary nail polish is prone to chipping. Furthermore, nail polishes incorporating glitter tend to leave an undesirable bumpy surface on the nail. Consequently, most people desiring a glitter effect choose go to a salon to have glitter professionally affixed to their nails using gel and/or acrylic resins.

While the use of gels and acrylic resins can produce a durable, smooth glitter effect on the nails, these methods also pose significant drawbacks. For instance, most gel resins are cured by ultraviolet light, and therefore require the use of a specialized ultraviolet lamp during application. Additionally, gel and acrylic resins create a permanent bond to the surface of the nail. As such, removal of the cured resin involves grinding the resin off the nail with powered equipment, which can damage the nail and limit the frequency with which resins may safely be applied and removed from the nail. Moreover, because the tools used in the application and removal of gel and acrylic resins are not common to most households, most people must make a costly trip to a nail salon each time they wish to apply or remove a gel or acrylic resin.

### BRIEF SUMMARY

A method of adhering ornamental particles to a nail, includes: applying a number of base layers of an adhesive lacquer to the nail; disposing a layer of the ornamental particles over the number of base layers of the adhesive lacquer; covering the layer of ornamental particles with a number of upper layers of the adhesive lacquer; and covering the number of upper layers of the adhesive lacquer with a number of layers of a sealant material.

A nail coating, includes: a number of base layers of an adhesive lacquer; a number of layers of ornamental particles disposed over the base layer; a number of upper layers of the adhesive lacquer disposed over the number of layers of particulate; and a number of layers of sealant material disposed over the number of upper layers of the adhesive lacquer.

A nail coating may comprise a layer of reflective particles encapsulated within a substantially clear adhesive lacquer, in which a first side of said encapsulation adheres to a nail and a second side of said encapsulation is covered with a high-gloss sealant material. In another example, the nail coating may comprise a wrap layer encapsulated within a substantially clear adhesive lacquer, in which a first side of said encapsulation adheres to a nail and a second side of said encapsulation is covered with a high-gloss sealant material. In this example, the wrap layer may be a sheet of vinyl shaped to fit over at least a portion of the top surface of the nail. The wrap layer may additionally include coloring or other types of designs which may be seen through the number of upper layers of the adhesive lacquer as well as the number of layers of sealant material.

### BRIEF DESCRIPTION OF THE DRAWINGS

The accompanying drawings illustrate various examples of the principles described herein and are a part of the specification. The illustrated examples do not limit the scope of the claims.

FIG. 1 is a flowchart showing a method of adhering ornamental particles to a nail according to one example of principles described herein.

FIGS. 2A through 2E are diagrams showing some points in the method of adhering ornamental particles to a nail shown in FIG. 1 according to one example of principles described herein.

FIG. 3 is a flowchart showing another method of adhering ornamental particles to a nail according to one example of principles described herein.

FIGS. 4A and 4B are cross-sectional diagrams of nail coatings having a layer of ornamental particles disposed therein according to one example of principles described herein.

FIG. 5 is a block diagram of a nail decoration kit according to one example of principles described herein.

Throughout the drawings, identical reference numbers designate similar, but not necessarily identical, elements.

### DETAILED DESCRIPTION

The present specification discloses methods and systems of adhering ornamental particles to a nail. As described above, glitter has been applied to nails using either polish containing the glitter or cured resins. Nail polishes containing glitter may result in an uneven nail surface finish that easily chips and may only last a few days. Cured resins may be expensive, difficult to remove, and cause damage to the natural nail.

By contrast, the methods and systems of the present specification allow for the adhesion of gutter and/or other ornamental particles to a nail in a manner resulting in a durable, long-lasting ornamental finish. The finish may be applied at home or at a nail salon, and may be easily removed with common solvents such as acetone- and ethyl acetate-based nail polish remover. Additionally, the methods and systems of the present specification advantageously do not require the use of an ultraviolet light to cure the finish, which can be time-consuming, expensive, and cause heat damage to the nail bed.

In the present specification and in the appended claims, the term “nail” is meant to be understood broadly as any human or mammalian nail.

Additionally, in the present specification and in the appended claims, the term “artificial nail” is meant to be understood broadly as any object shaped like a human or mammalian nail that is designed to be affixed on the surface of, or in place of, a human or mammalian nail.

Further, in the present specification and in the appended claims, the term “lacquer” is meant to be understood as any liquid that dries by solvent evaporation to produce a durable finish.

Even further, in the present specification and in the appended claims, the term “ornamental particles” is meant to be understood broadly as any object made of any type of material and of which may fit over the surface of either a nail or an artificial nail. In one example, the ornamental particle may be a number of glitter particles. In another example, the ornamental particle may be a thin sheet of vinyl which is sized to fit over at least a portion of the nail.

While the methods and systems of the present specification are described herein with specific respect to human nails, it should be understood that these principles may also be used to adhere ornamental particles to artificial nails with similar durability and aesthetic properties.

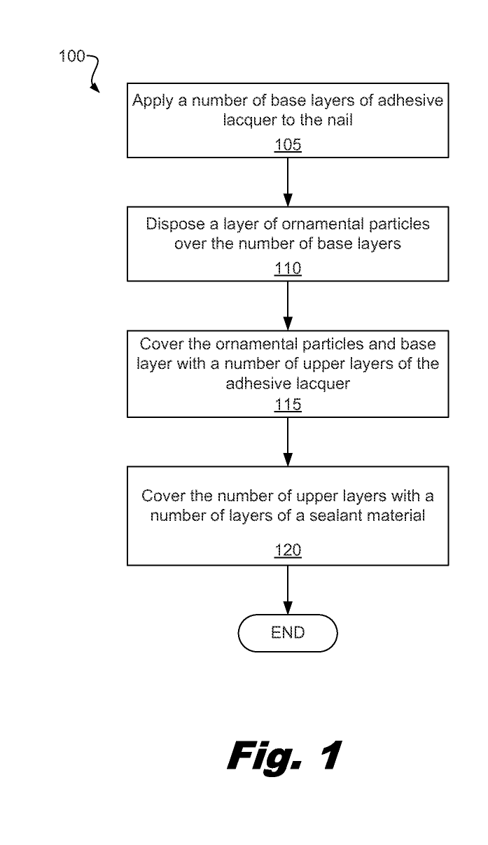

FIG. 1 is a flowchart showing a method (100) of adhering ornamental particles to a nail according to principles described herein. According to the method (100), a number of base layers of an adhesive lacquer is applied (block 105) to the nail. The adhesive lacquer functions as a base which adheres to and bonds with the surface of the nail. The adhesive lacquer may be a substantially clear, nonacrylic solution of nitrocellulose and/or other film former ingredients dissolved in solvents together with plasticizers. One formula of adhesive lacquer that has been found to be particularly suitable to the principles of the present specification is the following:

TABLE 1

| | Percentage by |
|---|---|
| Name | Weight |
| Ethyl Acetate | 24-29% |
| SD Alcohol 40B | 20-25% |
| Butyl Acetate | 17-21% |
| Nitrocellulose | 9-11% |
| Adipic Acid/Neopentyl | 5-7% |
| Glycol/Trimellitic | |
| Anhydride Copolymer | |
| SD Alcohol 3-A | 4-5% |
| N-Butyl Alcohol | 3-4% |
| Trimethyl Pentanyl | 2-3% |
| Diisobutyrate | |
| Triphenyl Phosphate | 2-3% |
| Camphor | 0.4-0.5% |
| Benzophenone-1 | 0.10-0.20% |
| Aloe Barbadensis | 5-7% |
| Extract | |
| Colorant | trace |

As shown in Table 1, the addition of an ultraviolet absorber (e.g., Benzophenone-1) and a botanical extract (e.g., Aloe Barbadensis extract) in the adhesive lacquer may provide strengthening and other therapeutic benefits to the nail. Additionally, these ingredients exhibit a synergistic strengthening effect when used together with a particular type of topcoat sealant.

Additionally, the inclusion of the ornamental particle or particles between the base layer and the number of adhesive upper layers of lacquer may provide further strength to the layers applied to the nail. Specifically, when the adhesive upper layers of lacquer set, the lacquer may conform to the surface details of the ornamental particles used in the ornamental particle layer. This may therefore permit any stress applied to the nail to be transmitted efficiently between the different materials. In one example, the ornamental particle may be roughened further to provide a stronger cohesive bond between it and the base and upper layers of adhesive lacquer. The cohesive bonding of the base, upper, and ornamental particle layers prevents chipping or cracking of the coating and thereby allows for a nail coating that will last relatively longer than other types of coatings.

Another example of a formula of adhesive lacquer that has been found to be suitable to the principles of the present specification is the following:

TABLE 2

| | Percentage |
|---|---|
| Name | by Weight |
| Butyl Actetate | 25.0-50.0% |
| Ethyl Acetate | 10.0-25.0% |
| Nitrocellulose | 10.0-25.0% |
| SD Alcohol 40-B | 5.0-10.0% |
| Adipic Acid/Neopentyl | 5.0-10.0% |
| Glycol/Trimellitic | |
| Isopropyl Alcohol | 5.0-10.0% |
| Trimethyl Pentanyl | 5.0-10.0% |
| Diisobutyrate | |
| n-Butyl Alcohol | 1.0-5.0% |
| Sucrose Benzoate | 1.0-5.0% |
| Stearalkonium | 0.1-1.0% |
| Hectorite | |
| Dimethicone | <0.1% |
| Epoxidized Soybean | <0.1% |
| Oil | |
| Benzophenone-1 | <0.1% |
| Aloe Barbadensis Leaf | <0.1% |
| Extract & Mineral OH | |
| Alumina [Nano] | <0.1% |
| Silica | <0.1% |
| Violet 2 (Color Index # | <0.1% |
| 60725) | |

As described above, the inclusion of an ornamental particle between the base layer having the chemical formula described in table 2 and the upper layer of lacquer provides a stronger cohesive bond throughout the entire nail coating. This prevents the nail from prematurely chipping or cracking while on the nail. Additionally, the various chemicals within the formulations of the base and upper lacquer layers may further provide chemical bonds so as to add more physical strength to the nail coating.

Yet another example of a formula of adhesive lacquer that has been found to be suitable to the principles of the present specification is the following:

TABLE 3

| | Percentage |
|---|---|
| Name | by Wieght |
| Ethyl Acetate | 24-29% |
| SD Alcohol 40B | 20-25% |
| Butyl Acetate | 17-21% |
| Nitrocellulose | 9-11% |
| Adipic Acid/Neopentyl | 5-7% |
| Glycol/Trimellitic | |
| Anhydride Copolymer | 0-25% |
| SD Alcohol 3-A | 4-5% |
| N-Butyl Alcohol | 3-4% |
| Trimethyl Pentanyl | 2-3% |
| Diisobutyrate | |
| Triphenyl Phosphate | 2-3% |
| Camphor | 0.40-0.50% |
| Benzophenone-1 | 0.10-0.20% |
| Aloe Barbadensis | 5-7% |
| Extract | |
| D&C Red No. 6 Barium | Quantity |
| Lake | sufficient |

The formulations described in tables 1-3 may include a number of solvents (i.e. ethyl acetate, SD alcohol 40B, Butyl Acetate, SD alcohol 3-A, and N-Butyl alcohol) at the various concentrations described therein. Additionally, a number of film formers (i.e. Nitrocellulose and Adipic Acid/Neopentyl Glycol/Trimellitic) at the various concentrations described. Even further, a number of plasticizers (i.e., trimethyl pentanyl diisobutyrate, triphenyl phosphate, camphor) may be added at the various concentrations described. Although tables 1-3 describe some examples of formulations that may be used for the base layer of the nail coating, these are merely examples and the chemicals used to form the base layer formulation may include some, all, or additional chemicals found through these examples. Additionally, the various chemicals include a range of each ingredient. In some examples, the range of the amount of these ingredients may be a sub-range of those disclosed.

The base layer of the adhesive lacquer performs the dual function of bonding to the nail and providing an adhesive substrate for the deposition of ornamental particles (e.g., glitter or vinyl layer). In some examples, the application of multiple coats of the adhesive lacquer to the nail may create a thicker, more durable, and smoother base layer. Once base layer of the adhesive lacquer has been applied (block 105) to the nail, a layer of ornamental particles is disposed (block 110) over the base layer. In one example, the layer of ornamental particles may be disposed (block 110) while the upper surface of the base layer is still wet. In certain examples, pressure may be applied to the ornamental particles to embed the ornamental particles within the base layer.

The layer of ornamental particles and base layer are then covered (block 115) with a number of upper layers of the adhesive lacquer. In certain examples, the number of upper layers of the adhesive lacquer is applied to the layer of ornamental particles after the base layer has been allowed to at least partially dry, thereby firmly adhering the layer of ornamental particles to the upper surface of the base layer. Once the number of upper layers of the adhesive lacquer have been applied and at least partially dried, the layer of ornamental particles becomes substantially encapsulated within the adhesive lacquer. At this point, the number of upper layers of adhesive lacquer may be covered with a number of layers of a sealant material. The sealant material may form a smoother upper surface over a rough upper surface of the upper layer of adhesive lacquer and form a protective coating over the upper layer.

In certain examples, the sealant material may be a high-gloss, high-sheen sealing lacquer with a smoothing property (i.e., the ability to create a smooth layer over a rough surface), such as a cellulose acetate butyrate solution that includes a number of acrylic copolymers. One formula of sealant material that has been found to be particularly suitable to the principles of the present specification is shown in Table 3, which lists the ingredients of the sealant material in descending order of weight by percentage:

TABLE 4

Name
Ethyl Acetate
Isopropyl Alcohol
Cellulose Acetate
Butyrate
Butyl Acetate
Propyl Acetate
Heptane
Acrylates Copolymer
Sucrose Benzoate
Triphenyl Phosphate
Etocrylene
Benzophenone-1
2,2-(2,5-
Triophenediyl) Bis(5-
Tert-Butyl
Benzoxazole)

Another formula of sealant material that has been found to be suitable to the principles of the present specification is shown in Table 5, which lists the ingredients of the sealant material as well as their weight by percentage:

TABLE 5

| | Percentage by |
|---|---|
| Name | Weight |
| Butyl Actetate | 25.0-50.0% |
| Ethyl Acetate | 10.0-25.0% |
| Nitrocellulose | 10.0-25.0% |
| SD Alcohol 40-B | 5.0-10.0% |
| Adipic Acid/Neopentyl Glycol/Trimellitic | 5.0-10.0% |
| Anhydride Copolymer | |
| n-Butyl Alcohol | 5.0-10.0% |
| Trimethyl Pentanyl Diisobutyrate | 5.0-10.0% |
| Isopropyl Alcohol | 5.0-10.0% |
| Sucrose Benzoate | 1.0-5.0% |
| Epoxidized Soybean Oil | <0.1% |
| Benzophenone-1 | <0.1% |
| Violet 2 (Color Index # 60725) | <0.1% |

The various chemicals described in Table 5 include a range of each ingredient. In some examples, the range of the amount of these ingredients may be a sub-range of those disclosed.

The sealant material may be formulated to be particularly fast drying. Because the adhesive lacquer may take a number of hours to fully dry and set, the fast-drying property of the sealing material may give the appearance that the nail is fully dry within a matter of minutes, in addition to imparting protection to the underlying layers of adhesive lacquer and ornamental particles as the nail coating fully sets. For example, it has been observed that by using the formulas for adhesive lacquer and sealant material given in Tables 1 and 2 to apply a nail coating according to the method (100) of FIG. 1, the upper surface of the nail coating may dry. Consequently, the surface of the nail will be substantially safe from the risk of sticking to foreign objects or smudging of the aesthetic effect created by the layers of adhesive lacquer and ornamental particle layers.

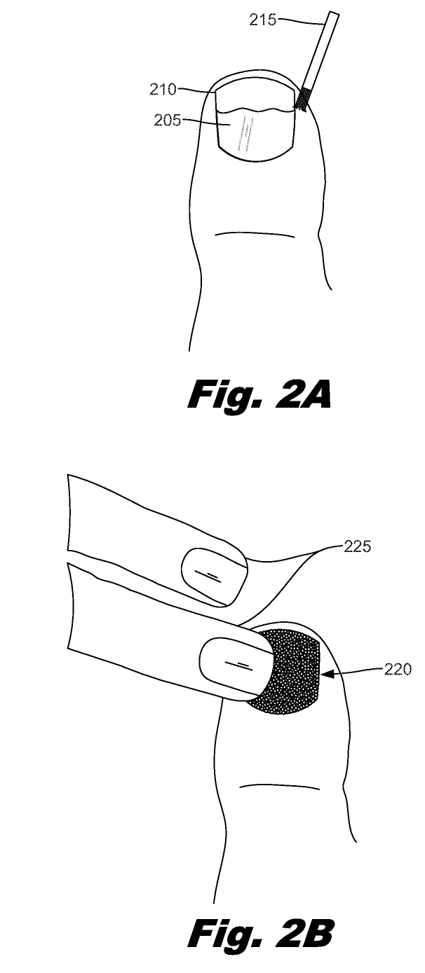

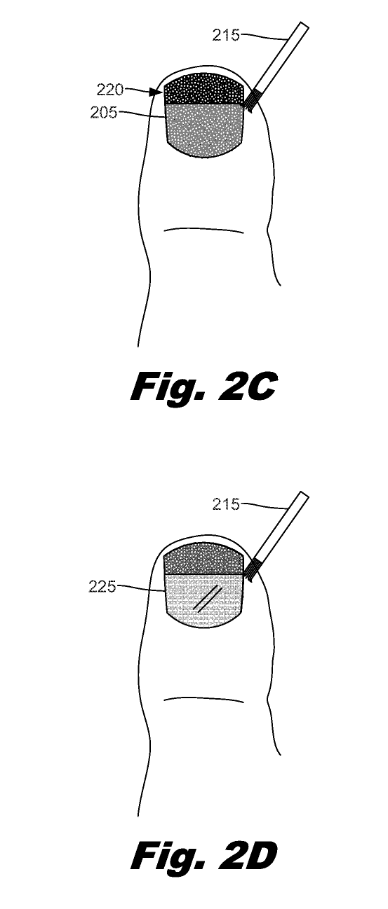

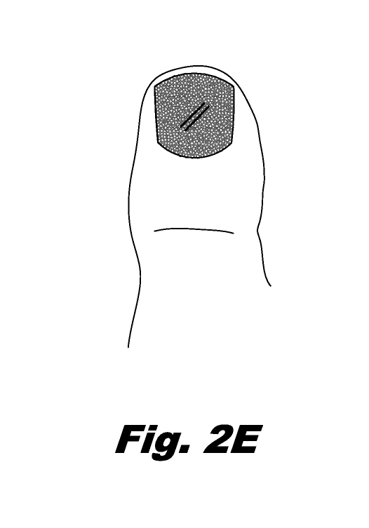

FIGS. 2A-2E are diagrams of various phases of the method (100) of FIG. 1. FIG. 2A shows the application of adhesive lacquer (205) to a nail (210) using a brush (215). FIG. 2B shows the application of pressure to a layer of ornamental particles (220) with fingers (225) to adhere the ornamental particles (220) to the upper surface of the base layer. FIG. 2C shows the application of the number of upper layers of adhesive lacquer (205) with a brush (215) to cover the ornamental particles (220) and base layer. FIG. 20 shows the application of the number of layers of sealant material (225) over the upper layer of adhesive lacquer with a brush (215). FIG. 2E shows the finished nail having a coating of a number of base layers of adhesive lacquer, a number of layers of ornamental particles, a number of upper layers of adhesive lacquer, and a number of layers of sealant. Because the adhesive lacquer and sealant material are substantially clear, the ornamental particles may be clearly visible on the nail. Additionally, the high-gloss sealant material may further add to the sheen of the finished nail in a visually pleasing manner.

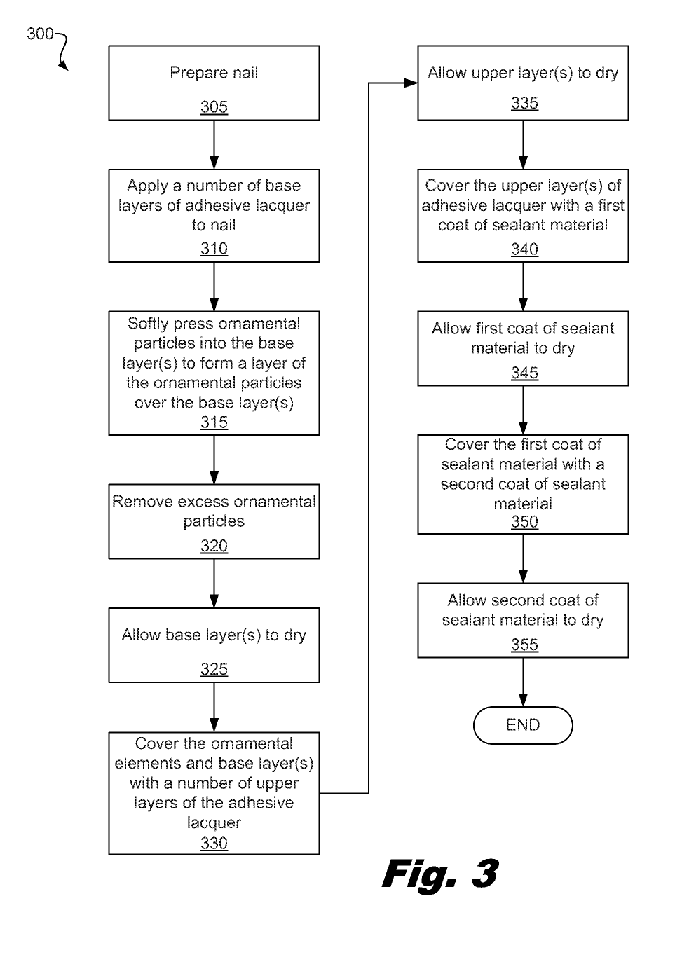

FIG. 3 is a flowchart of a more detailed example of a method (300) of adhering ornamental particles to a nail based on the principles described in FIGS. 1-2. The method (300) includes preparing (block 305) the nail. This preparation may include shaping the nail with a file, pushing back the cuticle of the nail, removing debris from around and under the nail, washing the nail bed, and allowing the nail to dry. In one example, the preparation does not include texturing or otherwise scrapping the top layer of the nail. As discussed above, the nail may be prepared in such a way so that subsequent removal of the coating, when desired, will not result in a nail plate that is scrapped and therefore unattractive. Additionally, because the process does not involve roughing the nail plate prior to the application of the coatings, the health of the nail may be preserved and the likelihood of infection due to the roughing process may be reduced or even eliminated.

A number of base layers of adhesive lacquer are applied (block 310) to the prepared nail, and ornamental particles such as glitter are softly pressed into the upper surface of the wet base layer to form (block 315) a layer of the ornamental particles over the base layer. Excess ornamental particles (i.e., ornamental particles that do not stick to the base layer) may be removed (block 320), and the base layer is allowed (block 325) to at least partially dry.

The ornamental elements and at least partially dry base layer(s) are then covered (block 330) with a number of upper layers of the adhesive lacquer, which are also allowed (block 335) to at least partially dry. The at least partially dry upper layer of adhesive lacquer is then covered (block 340) with a first coat of high-sheen, high-gloss sealant material, and the first coat of sealant material is allowed to dry (block 345). A second coat of the sealant material is applied (block 350) over the first coat of the sealant material, and the second coat of sealant is allowed (block 355) to dry, thereby completing the process. The adhesive lacquer, ornamental particles, and sealant material can all be removed from the nail using a common solvent, such as an acetone- or ethyl acetate-based nail polish remover.

The synergistic effect of the ingredients of the adhesive lacquer (e.g., Table 1) and the ingredients of the sealant material (e.g., Table 3) synergistically interact to produce a nail coating that is durable, long-lasting, and resistant to damage and changes in appearance (e.g., discoloration, scratches, streaks, etc.). In some cases, the resultant nail coating may last for months while preserving an attractive view of the ornamental particles.

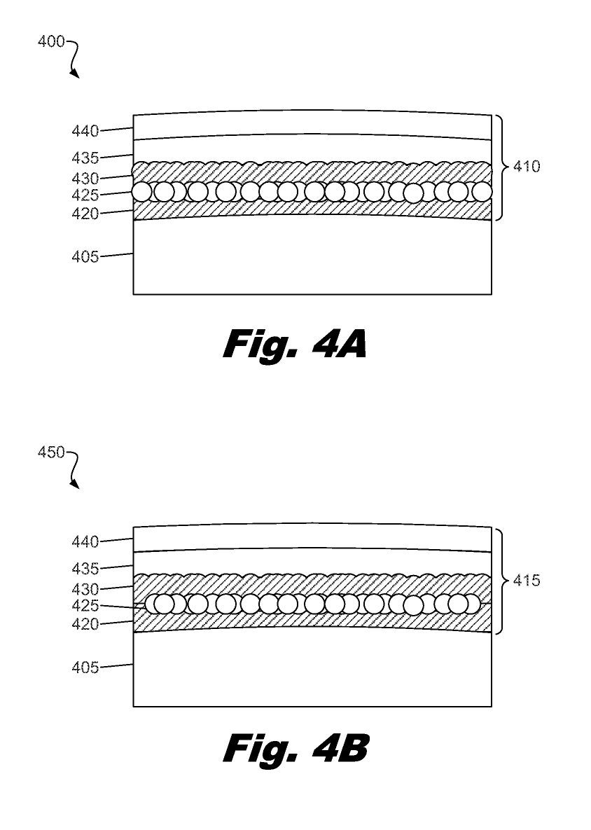

FIGS. 4A and 4B are cross-sectional diagrams of a nail (405) having a coating (410, 415) created consistent with the principles described above with respect to FIGS. 1-3. The coatings (410, 415) in FIGS. 4A and 4B are each made of up of four layers: a base layer (420) of an adhesive lacquer, a layer of ornamental particles (425), an upper layer (430) of adhesive lacquer, a first coat (435) of sealant material, and a second coat (440) of sealant material. As shown in FIGS. 4A and 4B, the placement of the ornamental particles (425) may, in some cases, give the upper layer (430) an uneven upper surface. The sealant lacquer may be designed to perform a smoothing function such that the upper surface of the coats (435, 440) of sealant material is smooth. FIG. 4B differs from FIG. 4A in that FIG. 4B shows an example in which the base and upper coats (420, 430) of adhesive lacquer extend around either side of the layer of ornamental particles (425), thereby fully encapsulating and providing lateral protection to the layer of ornamental particles (425).

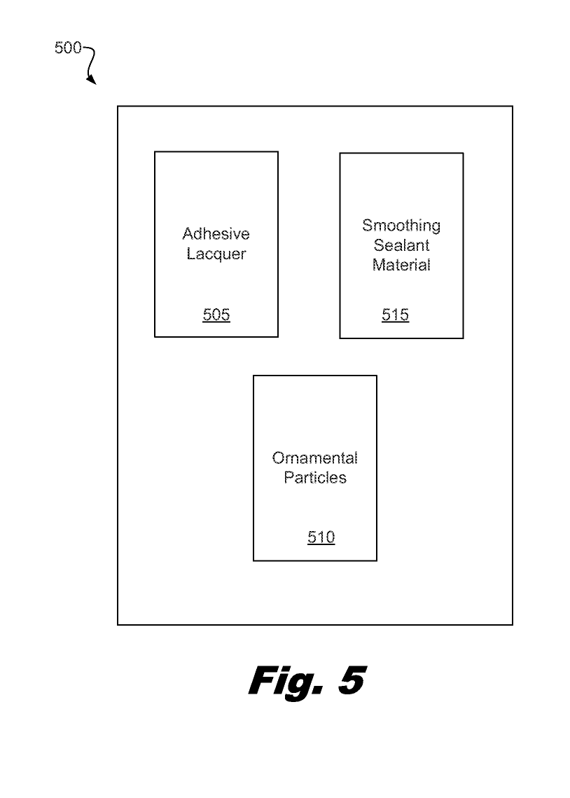

FIG. 5 is a block diagram of a nail decoration kit (500), in which an adhesive lacquer (505), a plurality of ornamental particles (510), and a smoothing sealant material are packaged together. The adhesive lacquer (505) may be substantially clear, nonacrylic lacquer based on a nitrocellulose solution, consistent with the explanations given previously in the present specification. The ornamental particles (510) may, in some examples, be reflective glitter. In another example, the ornamental particle may comprise a sheet of vinyl shaped to fit over at least a portion of the top surface of the nail. The smoothing sealant material may be a high-gloss, high-sheen, smoothing lacquer based on a cellulose acetate butyrate solution having a number of acrylic copolymers.

Having thus described the nail coating, decorating kit and method of adhering ornamental particles to a nail of the present application in detail and by reference to examples thereof, it will be apparent that modifications and variations are possible without departing from the scope of the nail coating, kit and method defined in the appended claims.

What is claimed is:

1. A method of adhering ornamental particles to a nail, said method comprising:
applying a number of base layers of an adhesive lacquer to said nail;
disposing a layer of said ornamental particles over said number of base layers of said adhesive lacquer;
covering said layer of ornamental particles with a number of upper layers of the adhesive lacquer; and
covering the upper layer of the adhesive lacquer with a number of layers of a sealant material.

2. The method of claim 1, in which said layer of said ornamental particles is disposed over said base layer while said base layer is wet.

3. The method of claim 1, further comprising applying pressure to said ornamental particles to embed said ornamental particles into said base layer.

4. The method of claim 1, further comprising allowing said base layer to dry before applying said upper layer.

5. The method of claim 1, further comprising allowing said upper layer to dry before applying said layer of sealant material.

6. The method of claim 1, in which said adhesive lacquer is a nonacrylic lacquer.

7. The method of claim 1, in which said adhesive lacquer is substantially clear.

8. The method of claim 1, in which said adhesive lacquer comprises a nitrocellulose solution.

9. The method of claim 1, in which said ornamental particles comprise glitter.

10. The method of claim 1, in which said upper layer of said adhesive lacquer comprises a substantially rough upper surface; and in which said layer of sealant comprises a substantially smooth surface.

11. The method of claim 1, in which said sealant material comprises a high-gloss, high-sheen lacquer.

12. The method of claim 1, in which said sealant material comprises a cellulose acetate butyrate solution comprising a number of acrylic copolymers.

13. A nail coating created by the method of claim 1.

14. A nail decoration kit, comprising:
an adhesive lacquer, ornamental particles, a sealant material, and tangible media comprising instructions for using said adhesive lacquer, ornamental particles, and sealant material to create a nail coating comprising:
a number of base layers of an adhesive lacquer;
a number of layers of ornamental particles disposed over said base layers; and
a number of upper layers of said adhesive lacquer disposed over said number of layers of ornamental particles; and
a number of layers of sealant material disposed over said number of upper layers of the adhesive lacquer.

15. The nail coating of claim 14, in which said adhesive lacquer comprises a dried nitrocellulose solution.

16. The nail coating of claim 14, in which said sealant comprises a high-gloss, high-sheen material.

17. The nail coating of claim 14, in which said sealant material comprises a dried cellulose acetate butyrate solution comprising a number of acrylic copolymers.

18. A nail coating comprising a layer of reflective particles encapsulated within a substantially clear adhesive lacquer, in which a first side of said encapsulation adheres to a nail and a second side of said encapsulation is covered with a high-gloss sealant material.

19. The nail coating of claim 18, in which said adhesive lacquer comprises a dried nitrocellulose solution.

20. The nail coating of claim 18, in which said sealant material comprises a dried cellulose acetate butyrate solution comprising a number of acrylic copolymers.
